(19) United States
(12) Patent Application Publication
(10) Pub. No.: US 2016/0151629 A1
(43) Pub. Date: Jun. 2, 2016

(54) COCHLEAR IMPLANT SYSTEM

(71) Applicant: ADVANCED BIONICS AG, Staefa (CH)

(72) Inventors: Josef Chalupper, Paunzhausen (DE); Patrick J. Boyle, Kent (GB)

(21) Appl. No.: 14/902,849

(22) PCT Filed: Jul. 5, 2013
(86) PCT No.: PCT/EP2013/064251

§ 371 Date: Jan. 4, 2016

(51) Int. Cl. A61N 1/36

(52) U.S. Cl. CPC A61N 1/36032

(57) ABSTRACT

There is provided a system comprising a device for neural stimulation of a cochlea of a patient's ipsilateral ear, a device for acoustic stimulation of the contralateral ear, and a fitting device for adjusting at least the neural stimulation device according to a perceptual behavioral response of the patient to combined neural stimulation of the cochlea at the ipsilateral ear and acoustic stimulation of the contralateral ear.

The invention relates to a system comprising a device for neural stimulation of the cochlea, a device for acoustic stimulation of the same ear or the other ear and a fitting device for individually adjusting the neural stimulation device to the patient.

Typically, cochlear implants comprise an electrode array for electrical stimulation of the cochlear at various stimulation sites determined by the position of the respective electrode. Systems for bimodal stimulation of the hearing comprise a cochlear implant at the ipsilateral ear and a device for acoustic stimulation of the ipsilateral ear or the contraletral ear. Systems with electric and acoustic stimulation of the same ear are also known as hybrid devices or EAS devices. In systems with contralateral acoustic stimulation the acoustic stimulation device typically is an (electro-acoustic) hearing aid.

For optimal fitting of such bimodal systems knowledge about the location of the electrodes of the electrode array with regard to the cochlea after surgery is an important prerequisite.

In principle, the electrode location could be determined via CT (computer tomography) scans. However, such a method would be expensive and would require an additional appointment for the patient in another clinical department, and also there would be an additional radiation dose which is difficult to justify except for a diagnostic test directly impacting the patient's health.

A more practical approach is to use behavioral pitch matching for determining the pitch and the electrode location. An example of such procedure is discussed in the article “Pitch comparison to an electrical stimulation of a cochlear implant and acoustic stimuli presented to a normal-hearing contralateral ear” by R. Carlyon et al., in JARO 11, 2010, pages 625 to 640, wherein either pure tones or filtered harmonic complexes are presented to the normal hearing ear as acoustic stimuli and electric stimuli are presented as biphasic pulse trains presented in monopolar mode to one electrode, with the acoustic stimuli and the electric stimuli being presented simultaneously or subsequently to the patient. Unfortunately, such pitch matching procedure is very tedious and unreliable.

According to the article “Contralateral masking in cochlear implant users with residual hearing in the non-implanted ear” by C. James et al., Audiology & Neuro-Otology 6, 2011, pages 87 to 97, threshold elevations for electrical stimulation probes were observed when acoustic contralateral maskers were presented; the acoustic masking signals were tones or narrow band noise signals.

US 2005/0261748 A1 relates to a fitting method for a hybrid device used by a patient having residual acoustic hearing capability at the ipsilateral ear, wherein the portion of the cochlea having residual acoustic hearing capability is determined by measuring the neural response to acoustic and/or electrical stimulation. Acoustic background noise, in particular narrow band background stimulus of a frequency substantially corresponding to the position of the tip electrode, is applied together with an electrical stimulus in order to determine from ECAP measurements which portion of the cochlear has residual acoustic hearing capability, with the ECAP measurements being used to determine a frequency-electrode position map.

US 2011/0238176 A1 likewise relates to a fitting method for a hybrid device, wherein a tonotopic response for the residual hearing of the ipsilateral cochlear is measured to obtain a place-frequency map, the CI implant is inserted according to the place-frequency map, and the position of the CI then is confirmed according to the measured place-frequency map via the measurement of the evoked neural response, such as the auditory brainstem response (ABR), to electrical stimulation of the CI and simultaneous acoustic stimulation. The acoustic stimulus is a customized chirp signal.

It is an object of the invention to provide for a bimodal stimulation system comprising a fitting device allowing for fast, easy, reliable and clinically appropriate determination of electrode positions after surgery for patients with residual hearing at the ipsilateral and/or contralateral ear. It is also an object to provide for a corresponding bimodal fitting method.

According to the invention, these objects are achieved by systems as defined in claims 1 and 3 respectively and methods as defined in claims 28 and 29, respectively.

The invention is beneficial in that by using a notch-type acoustic broadband masking signal for obtaining a perceptual behavioral response of the patient to synchronized neural stimulation of the ipsilateral cochlear with the probe neural stimulation signal and the acoustic stimulation of the contralateral or ipsilateral ear with the notch-type acoustic broadband masking signal, the perceived frequency of the neural stimulation sites can be determined in a fast, simple, reliable and clinically appropriate manner. In particular, such frequency determination by applying an acoustic masking signal having a notch frequency region is easier and more reliable than a pitch matching procedure in which the perception of the neural stimulus is compared to acoustic stimulation by a pure tone or a narrowband signal.

Preferred embodiments are defined in the dependent claims.

Hereinafter, the invention will be illustrated by reference to the attached drawings, wherein:

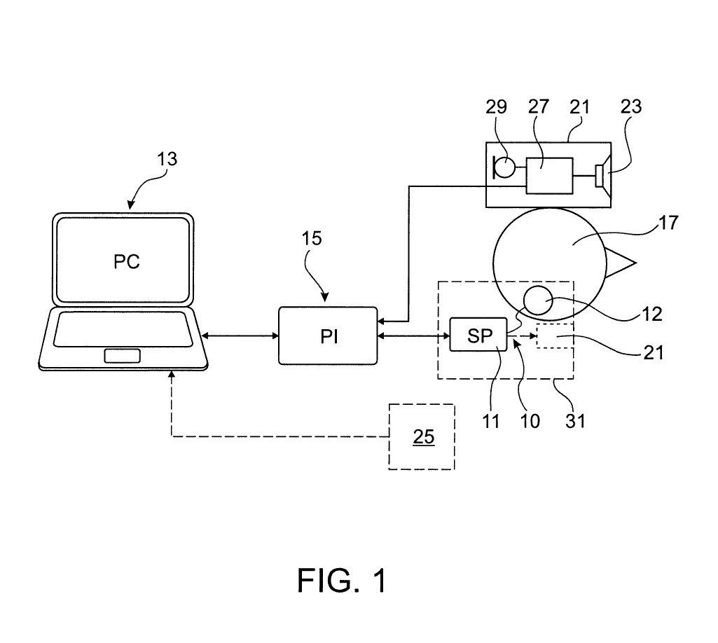

FIG. 1 is a schematic representation of an example of a system according to the invention;

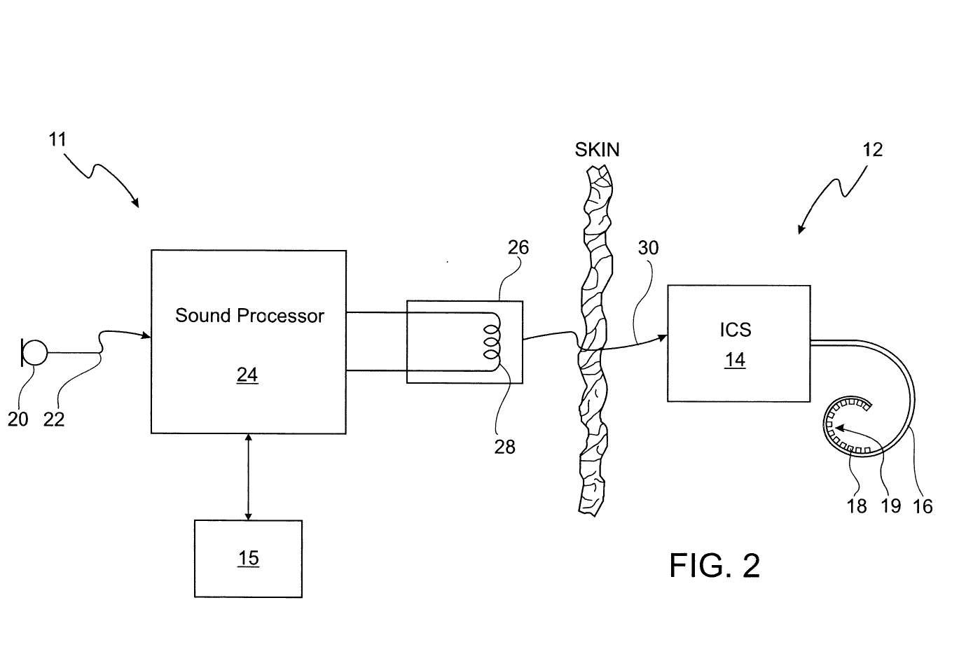

FIG. 2 is a schematic representation of an example of the CI device of FIG. 1;

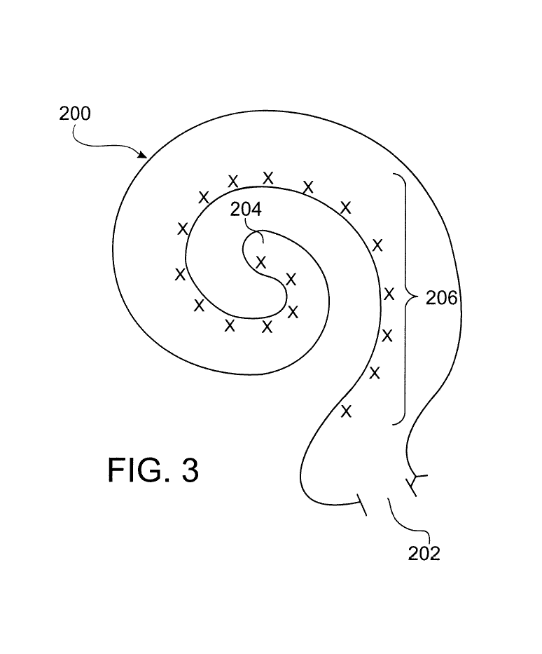

FIG. 3 is a schematic cross sectional view of a human cochlear with marked stimulation sites;

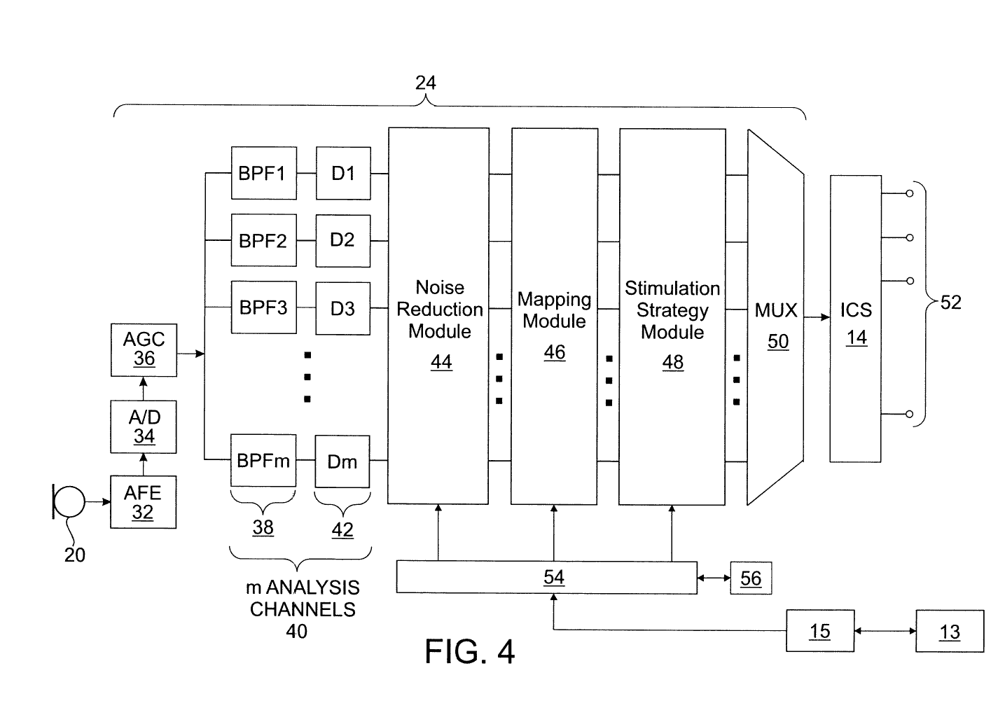

FIG. 4 is a block diagram of an example of the signal processing structure of a CI device to be used with the invention;

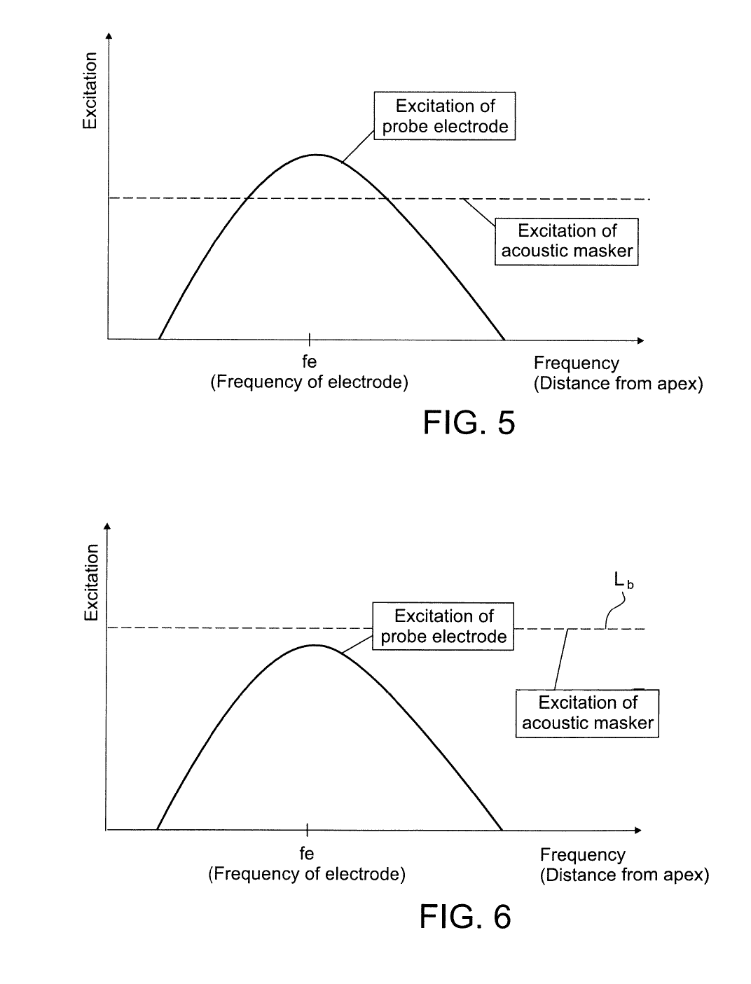

FIG. 5 is an example of the excitation at the ipsilateral ear as a function of frequency by combined stimulation with a probe neural stimulus and an acoustic broad band masking signal during a first step of a fitting procedure according to the invention, wherein the probe stimulus is still audible;

FIG. 6 is a diagram like FIG. 5, wherein however, the excitation level of the acoustic masking signal is increased to an extend that the probe signal is no longer audible;

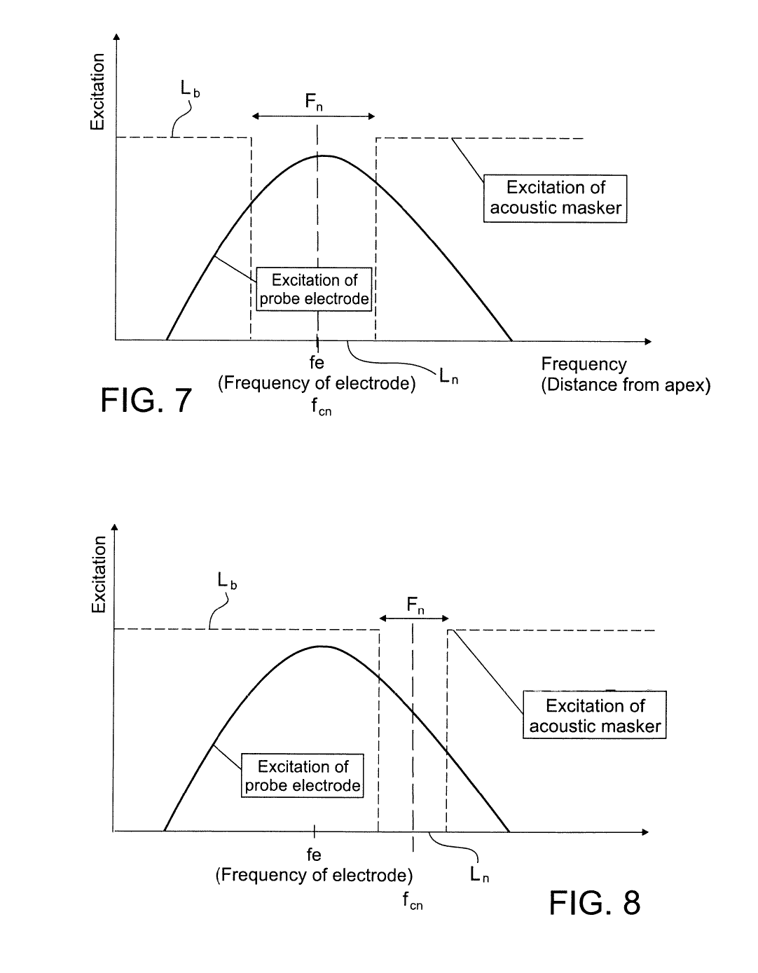

FIGS. 7 and 8 are diagrams like FIGS. 4 and 5, wherein however, notch-type broad band acoustic masking signals are applied, having different center frequencies of the notch region;

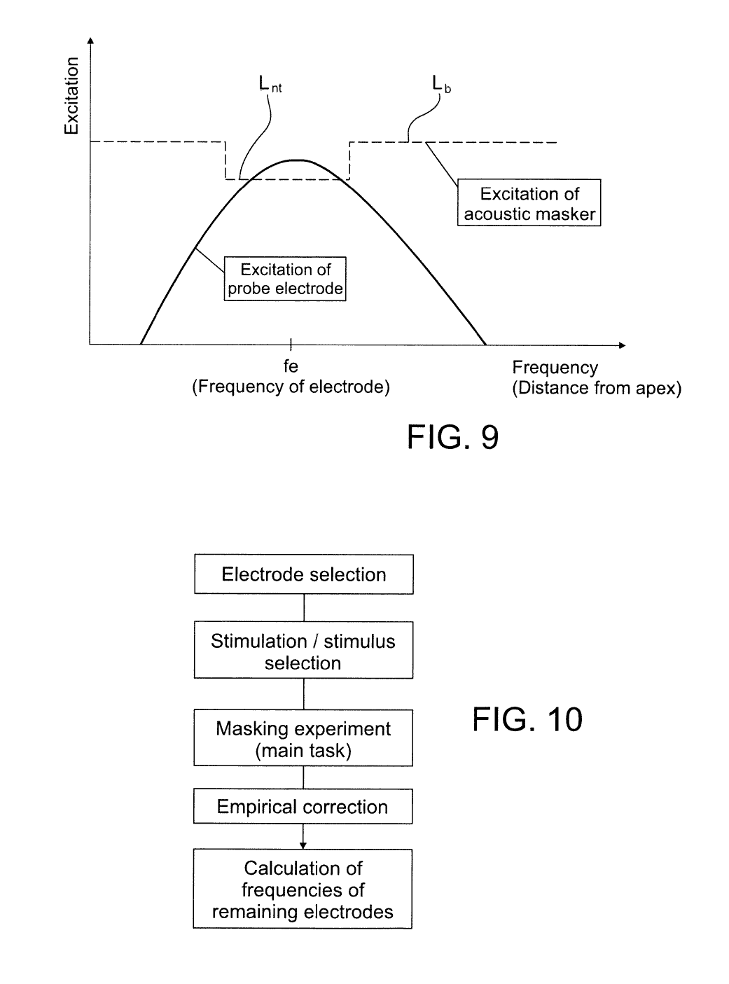

FIG. 9 is a diagram like FIGS. 5 to 8, wherein the level within the notch region of the notch-type broad band masking signal is increased until the probe signal is no longer audible; and

FIG. 10 is a flow chart of an example of a fitting method according to the invention.

FIG. 1 is a schematic representation of an example of a bimodal stimulation system according to the invention, comprising a fitting/programming unit 13, which may be implemented as a computer, a programming interface 15, a CI device 10 comprising a sound processing subsystem 11 and an implantable relation subsystem 12 and being worn by a patient 17 at the ipsilateral ear, and a hearing aid 21 worn at the contralateral ear and comprising a loudspeaker 23 for acoustic stimulation of the contralateral ear. The programming unit 13 communicates with the sound processing subsystem 11 and with the hearing aid 21 via the programming interface 15, which may be implemented as a wired or wireless connection.

The programming unit 13 serves to control the sound processing subsystem 11 of the CI device 10 such that probe neural stimulation signals are applied to the ipsilateral ear of the patient 17 via the stimulation subsystem 12 and to control the hearing aid 21 such that acoustic broadband masking signals are presented via the loudspeaker 23 to the contralateral ear of the patient 17 in a synchronized manner with regard to the probe neural stimulation. The perceptual behaviorial response of the patient 17 to the such synchronized stimulation is recorded by the programming unit 13 via a user interface, which may be part of the programming unit (such as the computer keyboard) or may be provided separately (as schematically indicated at 25 in FIG. 1), in order to determine the place-frequency map of the neural stimulation sites within the cochlea. Such place-frequency map then is used in programming the sound processing subsystem 11 in order to fit the CI device 10 and the hearing aid 21 as a bimodal system to the patient 17.

It is to be understood that the programming unit 13 is used with the CI device 10 and the hearing aid 21 only for adjustment/fitting, but not during normal operation of the CI device 10 and the hearing aid 21.

In case that the fitting/programming unit 13 is adapted to generate audio signals/stimulation signals on its own, the programming interface 15 may be replace by an audio interface for supplying the audio signals generated by the fitting/programming unit 13 to the CI device.

In FIG. 2 an example of the cochlear implant device 10 of the system of FIG. 1 is shown schematically. The sound processing sub-system 11 serves to detect or sense an audio signal and divide the audio signal into a plurality of analysis channels, each containing a frequency domain signal (or simply “signal”) representative of a distinct frequency portion of the audio signal. A signal level value and a noise level value are determined for each analysis channel by analyzing the respective frequency domain signal, and a noise reduction gain parameter is determined for each analysis channel as a function of the signal level value and the noise level value of the respective analysis channel. Noise reduction is applied to the frequency domain signal according to the noise reduction gain parameters to generate a noise reduced frequency domain signal. Stimulation parameters are generated based on the noise reduced frequency domain signal and are transmitted to the stimulation sub-system 12.

Stimulation sub-system 12 serves to generate and apply electrical stimulation (also referred to herein as “stimulation current” and/or “stimulation pulses”) to stimulation sites at the auditory nerve within the cochlear of a patient 17 in accordance with the stimulation parameters received from the sound processing sub-system 11. Electrical stimulation is provided to the patient 17 via a CI stimulation assembly 18 comprising a plurality of stimulation channels, wherein various known stimulation strategies, such as current steering stimulation or N-of-M stimulation, may be utilized.

As used herein, a “current steering stimulation strategy” is one in which weighted stimulation current is applied concurrently to two or more electrodes by an implantable cochlear stimulator in order to stimulate a stimulation site located in between areas associated with the two or more electrodes and thereby create a perception of a frequency in between the frequencies associated with the two or more electrodes, compensate for one or more disabled electrodes, and/or generate a target pitch that is outside a range of pitches associated with an array of electrodes.

As used herein, an “N-of-M stimulation strategy” is one in which stimulation current is only applied to N of M total stimulation channels during a particular stimulation frame, where N is less than M. An N-of-M stimulation strategy may be used to prevent irrelevant information contained within an audio signal from being presented to a CI user, achieve higher stimulation rates, minimize electrode interaction, and/or for any other reason as may serve a particular application.

The stimulation parameters may control various parameters of the electrical stimulation applied to a stimulation site including, but not limited to, frequency, pulse width, amplitude, waveform (e.g., square or sinusoidal), electrode polarity (i.e., anode-cathode assignment), location (i.e., which electrode pair or electrode group receives the stimulation current), burst pattern (e.g., burst on time and burst off time), duty cycle or burst repeat interval, spectral tilt, ramp on time, and ramp off time of the stimulation current that is applied to the stimulation site.

FIG. 3 illustrates a schematic structure of the human cochlea 200. As shown in FIG. 3, the cochlea 200 is in the shape of a spiral beginning at a base 202 and ending at an apex 204. Within the cochlea 200 resides auditory nerve tissue 206 which is organized within the cochlea 200 in a tonotopic manner. Low frequencies are encoded at the apex 204 of the cochlea 200 while high frequencies are encoded at the base 202. Hence, each location along the length of the cochlea 200 corresponds to a different perceived frequency. Stimulation subsystem 12 is configured to apply stimulation to different locations within the cochlea 200 (e.g., different locations along the auditory nerve tissue 206) to provide a sensation of hearing.

Returning to FIG. 2, sound processing subsystem 11 and stimulation subsystem 12 is configured to operate in accordance with one or more control parameters. These control parameters may be configured to specify one or more stimulation parameters, operating parameters, and/or any other parameter as may serve a particular application. Exemplary control parameters include, but are not limited to, most comfortable current levels (“M levels”), threshold current levels (“T levels”), dynamic range parameters, channel acoustic gain parameters, front and backend dynamic range parameters, current steering parameters, amplitude values, pulse rate values, pulse width values, polarity values, filter characteristics, and/or any other control parameter as may serve a particular application.

In the example shown in FIG. 2, the stimulation sub-system 12 comprises an implantable cochlear stimulator (“ICS”) 14, a lead 16 and the stimulation assembly 18 disposed on the lead 16. The stimulation assembly 18 comprises a plurality of “stimulation contacts” 19 for electrical stimulation of the auditory nerve. The lead 16 may be inserted within a duct of the cochlea in such a manner that the stimulation contacts 19 are in communication with one or more stimulation sites within the cochlea, i.e. the stimulation contacts 19 are adjacent to, in the general vicinity of in close proximity to, directly next to, or directly on the respective stimulation site.

In the example shown in FIG. 2, the sound processing sub-system 11 is designed as being located external to the patient 17; however, in alternative examples, at least one of the components of the sub-system 11 may be implantable.

In the example shown in FIG. 2, the sound processing sub-system 11 comprises a microphone 20 which captures audio signals from ambient sound, a microphone link 22, a sound processor 24 which receives audio signals from the microphone 20 via the link 22, and a headpiece 26 having a coil 28 disposed therein. The sound processor 24 is configured to process the captured audio signals in accordance with a selected sound processing strategy to generate appropriate stimulation parameters for controlling the ICS 14 and may include, or be implemented within, a behind-the-ear (BTE) unit or a portable speech processor (“PSP”). In the example of FIG. 2 the sound processor 24 is configured to transcutaneously transmit data (in particular data representative of one or more stimulation parameters) to the ICS 14 via a wireless transcutaneous communication link 30. The headpiece 26 may be affixed to the patient's head and positioned such that the coil 28 is communicatively coupled to the corresponding coil (not shown) included within the ICS 14 in order to establish the link 30. The link 30 may include a bidirectional communication link and/or one or more dedicated unidirectional communication links. According to an alternative embodiment, the sound processor 24 and the ICS 14 may be directly connected by wires.

In FIG. 4 a schematic example of a sound processor 24 is shown. The audio signals captured by the microphone 20 are amplified in an audio front end circuitry 32, with the amplified audio signal being converted to a digital signal by an analog-to-digital converter 34. The resulting digital signal is then subjected to automatic gain control using a suitable automatic gain control (AGC) unit 36.

After appropriate automatic gain control, the digital signal is subjected to a filterbank 38 comprising a plurality of filters F1 . . . Fm (for example, band-pass filters) which are configured to divide the digital signal into m analysis channels 40, each containing a signal representative of a distinct frequency portion of the audio signal sensed by the microphone 20. For example, such frequency filtering may be implemented by applying a Discrete Fourier Transform to the audio signal and then distribute the resulting frequency bins across the analysis channels 40.

The signals within each analysis channel 40 are input into an envelope detector 42 in order to determine the amount of energy contained within each of the signals within the analysis channels 40 and to estimate the noise within each channel. After envelope detection the signals within the analysis channels 40 may be input into a noise reduction module 44, wherein the signals are treated in a manner so as to reduce noise in the signal in order to enhance, for example, the intelligibility of speech by the patient. Examples of the noise reduction module 44 are described in WO 2011/032021 A1.

The optionally noise reduced signals are supplied to a mapping module 46 which serves to map the signals in the analysis channels 40 to the stimulation channels S1 . . . Sn. For example, signal levels of the noise reduced signals may be mapped to amplitude values used to define the electrical stimulation pulses that are applied to the patient 17 by the ICS 14 via M stimulation channels 52. For example, each of the m stimulation channels 52 may be associated to one of the stimulation contacts 19 or to a group of the stimulation contacts 19.

The sound processor 24 further comprises a stimulation strategy module 48 which serves to generate one or more stimulation parameters based on the noise reduced signals and in accordance with a certain stimulation strategy (which may be selected from a plurality of stimulation strategies). For example, stimulation strategy module 48 may generate stimulation parameters which direct the ICS 14 to generate and concurrently apply weighted stimulation current via a plurality 52 of the stimulation channels S1 . . . Sn in order to effectuate a current steering stimulation strategy. Additionally or alternatively the stimulation strategy module 48 may be configured to generate stimulation parameters which direct the ICS 14 to apply electrical stimulation via only a subset N of the stimulation channels 52 in order to effectuate an N-of-M stimulation strategy.

The sound processor 24 also comprises a multiplexer 50 which serves to serialize the stimulation parameters generated by the stimulation strategy module 48 so that they can be transmitted to the ICS 14 via the communication link 30, i.e. via the coil 28.

The sound processor 24 may operate in accordance with at least one control parameter which is set by a control unit 54. Such control parameters, which may be stored in a memory 56, may be the most comfortable listening current levels (MCL), also referred to as “M levels”, threshold current levels (also referred to as “T levels”), dynamic range parameters, channel acoustic gain parameters, front and back end dynamic range parameters, current steering parameters, amplitude values, pulse rate values, pulse width values, polarity values, the respective frequency range assigned to each electrode and/or filter characteristics. Examples of such auditory prosthesis devices, as described so far, can be found, for example, in WO 2011/032021 A1.

The programming unit 13 acts on the control unit 54 via the interface 15 for causing the ICS 14 and the electrode array 19 to apply a certain probe stimulus to the cochlear 200 as will be discussed in detail below.

The hearing aid 21 comprises a microphone arrangement 29 for capturing audio signals from ambient sound, an audio signal processing unit 27 for processing the captured audio signals and the loudspeaker 23 to which the processed audio signals are supplied to. The programming unit 13 acts, via the interface 15, on the audio signal processing unit 27 in order to cause the loudspeaker 23 to emit broadband masking signals supplied to the contralateral ear in a synchronized manner with regard to the probe stimulus applied by the CI device 10.

Hereinafter, an example of the fitting procedure will be described by reference to FIGS. 5 to 10.

As a first step, the electrode to be investigated is to be selected (usually it will be sufficient to determine the frequency/position of one electrode, since then the frequencies of the other electrodes can be estimated by applying an appropriate model, such as the Greenwood formulae or another procedure to calculate pitch from location or angle in the cochlea. The criteria for selecting the probe electrode may include discriminability, tonal perception, electrical impedance and/or ECAP patterns. Corresponding tests/experiments/measurements may be carried out for determining such parameters. For example, measurements using current steering may be conducted for estimating the sensitivity of each electrode with regard to pitch matching (and hence estimating the “independence” of different parts of the cochlea).

Further, the most appropriate neural stimulation method has to be selected, such as a “Simultaneous Analog Strategy” (SAS) or “Continuous Interleaved Sampling” (CIS). Also the most appropriate electrode coupling mode has to be selected, such as a multipolar or tripolar stimulation mode in order to provide for a “cleaner” probe signal for the masking experiment. The selected electrode may be activated in a pulsating manner (non-periodical/periodical) e.g. 500 ms on and 500 ms off.

The general goal of electrode and stimulation selection is to provide for a probe neural stimulus which is as “tonal” as possible.

As a next step, masking experiments are carried out, wherein an acoustic broadband masking signal and the probe neural stimulation signal are presented to the patient in a synchronized manner, the perceptual response of the patient is recorded and the acoustic broadband masking signal and/or the probe stimulation signal are varied in response to the recorded perceptual response by the patient, thereby implementing an iterative procedure. During such a procedure it is usually sufficient that the patient provides for feedback as to whether the probe stimulus is audible (i.e. is not masked by the acoustic signal) or is inaudible (i.e. is masked by the acoustic signal).

As a first step of the masking experiments, the acoustic signal is provided as a start broadband signal having an essentially constant excitation level over frequency (the frequency with regard to the neural stimulation corresponds to the distance from the apex of the cochlea). The experiment may start with a relatively low level of the acoustic signal which does not result in masking of the excitation of the probe stimulus. The level of the acoustic start broadband masking signal then is increased step by step until the probe stimulus becomes inaudible (see FIG. 6), thereby determining a masking threshold level of the start broadband noise masking signal. In this regard it does not matter that the acoustic masking signal is provided to the contralateral ear, since perception of the acoustic signal at the contralateral ear also has a masking effect with regard to neural stimulus perception at the ipsilateral ear. Preferably, the level of the start broadband masking signal is frequency-wise constant within 1 dB.

As an next step of the masking experiment, a notch acoustic broadband masking signal is applied which includes a notch frequency region having a noise level below the masking level of the probe stimulus, with the noise level outside the notch frequency region being above the masking level of probe stimulus.

An example of such notch masking signal is shown in FIG. 7, wherein the notch frequency region is indicated at Fₙ, the center frequency of the notch frequency region indicated at f_{cn}, the noise level outside the frequency region, which also may be referred to as a “base level”, is indicated at L_{b} and the noise level within the notch frequency region is indicated Lₙ. The base level outside the notch frequency region is derived from the masking threshold level determined by applying the start broadband signal illustrated in FIGS. 5 and 6. Preferably, the noise level outside the notch frequency region is frequency-wise relatively constant, preferably within 1 dB.

According to one example, the base level may equal the threshold masking level of the start broadband masking signal.

In the example of FIGS. 7 and 8 the noise level within the notch frequency region is zero, i.e.

there is no excitation of the ipsilateral ear within the notch frequency region. However, the noise level within notch frequency range also may be above zero as long as it is relatively low, so that no masking occurs within the notch frequency region.

The audibility of the probe stimulus depends on the position of the notch frequency region, i.e. on the center frequency thereof. In the example of FIG. 7, where the center frequency coincides with the frequency fₑ of the probe electrode, the probe stimulus would be fully audible. However, also in case of FIG. 8, where the center frequency of the notch frequency region has been shifted away from the frequency of the probe electrode, the probe stimulus still would be audible, since the probe stimulus not only provides for excitation at the frequency of the probe electrode but also in adjacent frequency regions, with the probe stimulus excitation level decreasing as a function of the distance from the frequency of the probe electrode.

The notch masking signals of the type shown in FIGS. 7 and 8 are used for determining a set of audible broadband noise signals (i.e. signals not resulting in masking of the probe neural stimulation signal) by stepwise shifting of the notch center frequency, with the signal set being parameterized by the respective notch center frequency fₑₙ.

Preferably, base noise level and the noise level in the notch frequency region are constant during determining that set of audible notch masking signals.

Preferably, the level within the notch frequency region is at least 10 dB less than the frequency-averaged base level (i.e. the level outside the notch frequency region) when determining the set of audible notch masking signals. Preferably, the slope at the edges of the notch frequency region is at least 30 dB/octave.

As a next step, for each member of the set of audible notch masking signals the level within the notch frequency region is readily or stepwise increased until a notch threshold level Lₙₜ is reached at which the probe stimulus becomes inaudible. Thus, for each member of the set of audible notch masking signals a respective notch threshold level Lₙₜ is obtained. The notch region center frequency of that member of the set of audible notch masking signals having the highest notch threshold level is assumed to correspond to the frequency fₑ of the probe electrode in order to obtain the value of the frequency fₑ of the probe electrode as the result of the masking experiments.

In some cases it may be appropriate to apply empirical corrections to the measurement results, so that the electrode frequency is derived from the notch region center frequency of the notch masking signal having the highest notch threshold level.

In the above described masking experiments, the masking threshold levels may be determined as a standard audiometry, with the patient, for example, pressing or releasing a button when the probe signal is no longer audible.

Since the audibility of the probe signal with regard to the masking signal only depends on the relative excitation levels, the above described procedure may be modified by keeping the level of the masking signal constant while varying the level of the probe stimulus. In this case, the level of the masking signal within the notch frequency region would be kept constant for each member of the set of audible notch masking signals, while the level of the probe stimulus is decreased stepwise. In the next step, that member of the set of audible notch masking signals would be taken as the “winner” for which the probe stimulus becomes inaudible at the lowest probe stimulus level. Similarly, also the step illustrated in FIGS. 5 and 6, wherein the base level of the masking signal is determined, may be modified in such a manner that the level of the masking signal is kept constant, while the level of the probe stimulus is gradually or stepwise reduced until the probe stimulus becomes inaudible. The level at which the probe stimulus becomes inaudible then would be used as the level of the probe stimulus for the shifting experiments, wherein the set of audible notch masking signals is determined (since the part of the procedure relating to the shifting of the notch center frequency does not involve any level changes, it may be the same for both variants).

In general, in the parts of the procedure involving level changes the relevant measure is the ratio of the probe stimulus level and the acoustic masking level.

Finally, the frequencies of the other electrodes may be estimated from the determined frequency of the probe electrode by applying a suitable model such as the Greenwood formulae or UCSF mapping.

According to a variant, the acoustic masking signals may be provided to the ipsilateral ear rather than to the contralateral ear. hi this case, the device worn at the ipsilateral ear may be a hybrid device providing both for electrical and acoustic stimulation of the ipsilateral ear, as indicated in dashed lines in FIG. 1 at 31.

According to a further variant, the neural stimulation may include optical stimulation of the cochlea in addition to or instead of the above described electrical stimulation, i.e. in this case an optical stimulus may be applied at the stimulation site in addition to or instead of an electrical stimulus.

1. A system comprising a device for neural stimulation of a cochlea of a patient's ipsilateral ear, a device for acoustic stimulation of the contralateral ear, and a fitting device for adjusting at least the neural stimulation device according to a perceptual behavioral response of the patient to combined neural stimulation of the cochlea at the ipsilateral ear and acoustic stimulation of the contralateral ear;
the neural stimulation device comprising means for providing an input audio signal; a sound processor for generating a neural stimulation signal from the input audio signal; and a cochlear implant stimulation arrangement comprising a plurality of stimulation channels for stimulating the cochlea at various stimulation sites according to a neural stimulation signal, with each stimulation channel being attributed to a certain one of the stimulation sites;
the acoustic stimulation device comprising a loudspeaker to be worn at the contralateral ear or in at least in part the ear canal of the contralateral ear for acoustically stimulating the contralateral ear according to an input audio signal,
the fitting device comprising
a signal generator cooperating with the neural stimulation device and with the acoustic stimulation device in order to generate, in a synchronized manner, a probe neural stimulation signal to be supplied to the cochlear implant stimulation arrangement for causing stimulation of the cochlea within a region around a selected one of the stimulation sites and a notch acoustic broadband masking signal be supplied to the loudspeaker, with the notch acoustic broadband masking signal including a notch frequency region having a noise level below a masking level at which masking of the probe neural stimulation signal begins and with the noise level outside the notch frequency region being above the masking level,
a unit for recording the perceptual behavioral response of the patient to the synchronized neural stimulation of the cochlea with the probe neural stimulation signal and the notch acoustic broadband masking signal, and
a unit for programming the neural stimulation device according to the recorded perceptual response.

2. The system of claim 1, wherein the acoustic stimulation device is a hearing aid to be worn at the contralateral side of the patient's head.

3. A system comprising a device for neural stimulation of a cochlea of a patient's ipsilateral ear, a device for acoustic stimulation of the ipsilateral ear, and a fitting device for adjusting at least the neural stimulation device according to the perceptual behavioral response of the patient to combined neural stimulation of the cochlea at the ipsilateral ear and acoustic stimulation of the ipsilateral ear;
the neural stimulation device comprising means for providing an input audio signal; a sound processor for generating a neural stimulation signal from the input audio signal; and
a cochlear implant stimulation arrangement comprising a plurality of stimulation channels for stimulating the cochlea at various stimulation sites according to a neural stimulation signal, with each stimulation channel being attributed to a certain one of the stimulation sites;
the acoustic stimulation device comprising a loudspeaker to be worn at the ipsilateral ear or in at least in part the ear canal of the ipsilateral ear for acoustically stimulating the ipsilateral ear according to an input audio signal,
the fitting device comprising
a signal generator cooperating with the neural stimulation device and with the acoustic stimulation device in order to generate, in a synchronized manner, a probe neural stimulation signal to be supplied to the cochlear implant stimulation arrangement for causing stimulation of the cochlea within a region around a selected one of the stimulation sites and a notch acoustic broadband masking signal to be supplied to the loudspeaker, with the notch acoustic broadband masking signal including a notch frequency region having a noise level below a masking level at which masking of the probe neural stimulation signal begins and with the noise level outside the notch frequency region being above the masking level
a unit for recording the perceptual behavioral response of the patient to the synchronized neural stimulation of the cochlea with the probe neural stimulation signal and the notch acoustic broadband masking signal, and
a unit for programming the neural stimulation device according to the recorded perceptual response.

4. The system of claim 3, wherein the neural stimulation device and the acoustic stimulation device are integrated within a hybrid device to be worn at the ispsilateral ear.

5. The system of claim 1, wherein the fitting device is adapted to generate the notch acoustic broadband masking signal and/or the probe neural stimulation signal in a variable manner responsive to the perceptual response by the patient.

6. The system of claim 5, wherein the fitting device is adapted to systematically vary a first parameter of the notch acoustic broadband masking signal until masking of the probe neural stimulation signal occurs.

7. The system of claim 6, wherein the fitting device is adapted to systematically vary, based on the results of the variation of the first parameter, a second parameter of the notch acoustic broadband masking signal and/or the level of the probe neural stimulation signal until masking of the probe neural stimulation signal occurs.

8. The system of claim 7, wherein the first parameter is the center frequency of the notch region and the second parameter is the noise level at the center frequency of the notch region.

9. The system of claim 8, wherein the frequency of the selected stimulation site is determined from the center frequency of the notch frequency region of that notch acoustic broadband masking signal which has the highest noise level at the center frequency of the notch frequency region at which masking of the probe neural stimulation signal begins or which has the lowest level of the probe neural stimulation signal at which masking of the probe neural stimulation signal begins.

10. The system of claim 1, wherein the notch acoustic broadband masking signal has a frequency-wise relatively constant base level outside the notch frequency region.

11. The system of claim 10, wherein the base level outside the notch frequency region is frequency-wise constant within 1 dB.

12. The system of claim 10, wherein the fitting device is adapted to determine a masking threshold by gradually increasing the level of a start broadband noise masking signal without a notch frequency region, thereby determining a threshold level of the start broadband noise masking signal at which the probe neural stimulation signal becomes inaudible due to masking in order to determine the base level of the notch broadband noise masking signal from the threshold level.

13. The system of claim 12, wherein the base level equals the threshold level.

14. The system of claim 12, wherein the level of the start broadband noise masking signal is frequency-wise constant within 1 dB.

15. The system of claim 10, wherein the fitting device is adapted to shift the notch center frequency of the notch broadband noise signal in a stepwise manner in order to determine a set of audible notch broadband noise signals not resulting in masking of the probe neural stimulation signal, with the set of audible notch broadband noise signals being parametrized by the respective notch center frequency.

16. The system of claim 15, wherein the base level and the level in the notch region are kept constant during determining said set of audible notch broadband noise signals.

17. The system of claim 16, wherein the level in the center of the notch frequency region is at least 10 dB less than the frequency-averaged base level during determining said set of audible notch broadband noise signals.

18. The system of claim 12, wherein the slope at the edges of the notch frequency region is at least 30 dB/octave.

19. The system of claim 15, wherein the fitting device is adapted to gradually increase the level within the notch frequency region of each member of said set of audible notch broadband noise signals until a notch threshold level is reached at which the probe neural stimulation signal becomes inaudible, wherein the center frequency of the notch frequency region of that member of said set of audible notch broadband noise signals having the highest notch threshold level is taken for determining the frequency of the selected stimulation site.

20. The system of claim 19, wherein the fitting device is adapted to estimate the frequencies of the other stimulation sites by applying a model.

21. The system of claim 20, wherein said model includes applying Greenwood formulae.

22. The system of claim, wherein the level of the probe neural stimulation signal is kept constant.

23. The system of claim 15, wherein the fitting device is adapted to gradually decrease the level of the probe neural stimulation signal for each member of said set of audible notch broadband noise signals until a notch threshold probe signal level is reached at which the probe neural stimulation signal becomes inaudible, wherein the center frequency of the notch frequency region of that member of said set of audible notch broadband noise signals having the lowest notch threshold probe signal level is taken for determining the frequency of the selected stimulation site.

24. The system of claim 1, wherein the cochlear implant stimulation arrangement comprises a plurality of electrodes for electrical stimulation of the cochlea, with each electrode forming one of the stimulation sites.

25. The system of claim 24, wherein the fitting device is adapted to cause the electrode of the selected stimulation site being activated in a pulsating manner.

26. The system of claim 24, wherein the fitting device is adapted to cause the cochlear implant stimulation arrangement to apply the probe neural stimulation signal via multipolar electrode coupling.

27. The system of claim 1, wherein the fitting device is implemented by a computer device communicating with the neural stimulation device and with the acoustic stimulation device via a programming interface.

28. A method of individually adjusting a device for neural stimulation of a patient's cochlea of the ipsilateral ear the according to an input audio signal, the device comprising a sound processor for generating a neural stimulation signal from the input audio signal and a cochlear implant stimulation arrangement comprising a plurality of stimulation channels for stimulating the cochlea at various stimulation sites according to a neural stimulation signal, with each stimulation channel being attributed to a certain one of the stimulation sites, the method comprising:
selecting one of the stimulation sites;
generating, by a fitting device cooperating with the neural stimulation device and a device comprising a loudspeaker worn at the contralateral ear or in at least in part the ear canal of the contralateral ear for acoustic stimulation of the contralateral ear, in a synchronized manner, a probe neural stimulation signal supplied to the cochlear implant stimulation arrangement for causing stimulation of the cochlea within a region around said selected stimulation site and a notch acoustic broadband masking signal supplied to the loudspeaker, with the notch acoustic broadband masking signal including a notch frequency region having a noise level below a masking level at which masking of the probe neural stimulation signal begins and with the noise level outside the notch frequency region being above the masking level;
recording a perceptual behavioral response of the patient to the synchronized neural stimulation of the cochlea with probe neural stimulation signal notch acoustic broadband masking signal;
determining the frequency of the selected stimulation site from the recorded perceptual response; and
programming the neural stimulation device according to the determined frequency of the selected stimulation site.

29. A method of individually adjusting a device for neural stimulation of a patient's cochlea of the ipsilateral ear the according to an input audio signal, the device comprising a sound processor for generating a neural stimulation signal from the input audio signal and a cochlear implant stimulation arrangement comprising a plurality of stimulation channels for stimulating the cochlea at various stimulation sites according to a neural stimulation signal, with each stimulation channel being attributed to a certain one of the stimulation sites, the method comprising:
selecting one of the stimulation sites;
generating, by a fitting device cooperating with the neural stimulation device and a device comprising a loudspeaker worn at the contralateral ear or in at least in part the ear canal of the contralateral ear for acoustic stimulation of the ipsilateral ear, in a synchronized manner, a probe neural stimulation signal supplied to the cochlear implant stimulation arrangement for causing stimulation of the cochlea within a region around said selected stimulation site and a notch acoustic broadband masking signal supplied to the loudspeaker, with the notch acoustic broadband masking signal including a notch frequency region having a noise level below a masking level at which masking of the probe neural stimulation signal begins and with the noise level outside the notch frequency region being above the masking level;
recording a perceptual behavioral response of the patient to the synchronized neural stimulation of the cochlea with probe neural stimulation signal notch acoustic broadband masking signal;
determining the frequency of the selected stimulation site from the recorded perceptual response; and
programming the neural stimulation device according to the determined frequency of the selected stimulation site.

30. The method of claim 28, wherein the notch acoustic broadband masking signal and/or the probe neural stimulation signal is/are generated in a variable manner responsive to a perceptual response by the patient.

31. The method of claim 30, wherein steps and are repeated with different notch acoustic broadband masking signals wherein at least one parameter of the notch acoustic broadband masking signal is systematically varied.

32. The method of claim 31, wherein one parameter of said at least one parameter is the center frequency of the notch region.

33. The method of claim 32, wherein one parameter of said at least one parameter is the level of at the center frequency of the notch region.

34. The method of claim 29, wherein estimating the frequencies of the other stimulation sites from the determined frequency of the selected stimulation site by applying a model and using the estimated frequencies in the programming of the neural stimulation device.

35. The method of claim 28, wherein the stimulation site is selected based on parameter measurements including at least one of electrode impedance, NRI pattern, tonal perception, and discriminability.
